(19) United States
(12) Patent Application Publication
(10) Pub. No.: US 2026/0204360 A1
(43) Pub. Date: Jul. 16, 2026

(54) LEARNING DEVICE, TRAINED MODEL, PREDICTION DEVICE, PREDICTION PROGRAM, AND PREDICTION METHOD

(71) Applicant: ENEOS Corporation, Tokyo (JP)

(72) Inventor: Tasuku ONODERA, Tokyo (JP)

(21) Appl. No.: 19/135,525

(22) PCT Filed: Nov. 24, 2023
(86) PCT No.: PCT/JP2023/042258

§ 371 Date: Jun. 4, 2025

(30) Foreign Application Priority Data
Dec. 14, 2022 (JP) 2022-199613

(51) Int. Cl. G16C 20/30; G16C 10/00; G16C 20/70

(52) U.S. Cl. CPC G16C 20/30; G16C 10/00; G16C 20/70

(57) ABSTRACT

A learning device includes a training part configured to generate a trained model through training using a training data set in which molecules of interest, which are molecules included in a base oil that is liquid at normal temperature of 20° C. and normal pressure of 1.013×10⁵ Pa, and a feature indicating an intermolecular space between the molecules of interest in a simulation cell having a freely chosen parallelepiped shape are associated with each other.

### TECHNICAL FIELD

The present invention relates to a learning device, a trained model, a prediction device, a prediction program, and a prediction method.

### BACKGROUND ART

In an industrial product provided with industrial machines, such as an internal combustion engine, hydraulic machine, compression machine, turbine, gear element, bearing, refrigerator, etc., in order to allow these machines to operate smoothly, various lubricants such as engine oil, hydraulic oil, compressor oil, turbine oil, gear oil, and refrigerating machine oil are used.

A lubricant mainly includes one or more base oils and one or more additives. As such a lubricant, for example, a lubricant composition containing an ester-based base oil and an additive for a lubricant, and the like are disclosed (for example, see Patent Document 1).

### RELATED ART DOCUMENTS

Patent Document 1: Japanese Unexamined Patent Application Publication No. 2022-158124

### SUMMARY OF THE INVENTION

### Problems to be Solved by the Invention

To allow a lubricant to exhibit desired lubricating performance, it is important to select a base oil that does not inhibit the effect of an additive or further enhances the effect of the additive. As a method of selecting a base oil, for example, there is a method of analyzing the structure of an oil film formed of the base oil by molecular simulation for each molecule constituting the base oil.

Specifically, as described in “Understanding the effect of the base oil on the physical adsorption process of organic additives using molecular dynamics” (M. Konishi and H. Washizu, Tribology International, 2020, vol. 149, article 105568), for example, there is a molecular dynamics simulation method, with which a diffusion behavior of an additive in a liquid phase of a base oil and an adsorption time of the additive to the surface of a metal plate are discussed, using an atomic/molecular scale model in which an additive is disposed in a liquid phase of a base oil and sandwiched between metal plates.

However, with this method, since it is necessary to perform complicated calculations, such as modeling and post-analysis, it takes a very long time to perform calculations, such as setting of analysis conditions, and it is not realistic to perform calculation for all molecules constituting a base oil. For example, in the case of analyzing the structure of an oil film formed of a base oil by molecular simulation for one type of base oil, it may take several days to several months until the calculation is completed. Therefore, a device for efficiently selecting a base oil that maximizes the effect of additives has been desired.

An objective of one aspect of the present invention is to shorten time required for selecting a base oil that brings out the effect of an additive.

### Means for Solving the Problem

One aspect of the present invention is a learning device including a training part configured to generate a trained model through training using a training data set in which molecules of interest, which are molecules included in a base oil that is liquid at normal temperature of 20° C. and normal pressure of 1.013×10⁵ Pa, and a feature indicating an intermolecular space between the molecules of interest in a simulation cell having a freely chosen parallelepiped shape are associated with each other.

Another aspect of the present invention is a trained model generated using a training data set in which molecules of interest, which are molecules included in a base oil that is liquid at normal temperature of 20° C. and normal pressure of 1.013×10⁵ Pa, and a feature indicating an intermolecular space between the molecules of interest in a simulation cell having a freely chosen parallelepiped shape are associated with each other.

Another aspect of the present invention is a prediction device including an acquirer configured to acquire molecules included in a base oil that is liquid at normal temperature of 20° C. and normal pressure of 1.013×10⁵ Pa as molecules of interest; and a predictor configured to predict a degree of density of an oil film formed of the molecules of interest using a trained model generated using a training data set in which the molecules of interest and a feature indicating an intermolecular space between the molecules of interest in a simulation cell having a freely chosen parallelepiped shape are associated with each other.

Another aspect of the present invention is a prediction program for causing a computer to execute: an acquisition step of acquiring molecules included in a base oil that is liquid at normal temperature of 20° C. and normal pressure of 1.013×10⁵ Pa as molecules of interest; and a prediction step of predicting a degree of density of an oil film formed of the molecules of interest using a trained model generated using a training data set in which the molecules of interest and a feature indicating an intermolecular space between the molecules of interest in a simulation cell having a freely chosen parallelepiped shape are associated with each other.

Another aspect of the present invention is a prediction method in which a computer is configured to execute: an acquisition step of acquiring molecules included in a base oil that is liquid at normal temperature of 20° C. and normal pressure of 1.013×10⁵ Pa as molecules of interest; a prediction step of predicting a degree of density of an oil film formed of the molecules of interest using a trained model generated using a training data set in which the molecules of interest and a feature indicating an intermolecular space between the molecules of interest in a simulation cell having a freely chosen parallelepiped shape are associated with each other.

### Effects of the Invention

One aspect of the present invention shortens time required for selecting a base oil that brings out the effect of an additive.

### BRIEF DESCRIPTION OF THE DRAWINGS

FIG. 1 is a block diagram illustrating a schematic configuration of a learning device according to an embodiment of the present invention.

FIG. 2 is a diagram illustrating an example of a table in which structural formulae (SMILES) are described.

FIG. 3 is an explanatory diagram illustrating a difference in adsorption states of an additive depending on a difference in a degree of density of an oil film.

FIG. 4 is an example of a functional block diagram illustrating a configuration of a device for calculating a feature.

FIG. 5 is a diagram illustrating an example of creating a three-dimensional structure from a single-molecule structure of molecules of interest.

FIG. 6 is a diagram illustrating an example of creating a liquid structure from the three-dimensional structure of the molecules of interest.

FIG. 7 is a diagram illustrating an example of a distance between two molecules.

FIG. 8 is a diagram illustrating an example of a radial distribution function.

FIG. 9 is a diagram illustrating an example of a radial distribution function of a common normal alkane liquid.

FIG. 10 is a diagram illustrating an example of a radial distribution function focusing only on an atomic distance between molecules in FIG. 9.

FIG. 11 is a functional block diagram illustrating a configuration of a prediction device.

FIG. 12 is a diagram illustrating an example of accuracy of predicted values predicted by a trained model.

FIG. 13 is a diagram illustrating an example of accuracy of another set of predicted values predicted by a trained model.

FIG. 14 is a diagram illustrating an example of a relationship between predicted values of two features.

FIG. 15 is a block diagram illustrating a hardware configuration of the prediction device.

FIG. 16 is a flowchart illustrating a training method according to an embodiment of the present invention.

FIG. 17 is a flowchart illustrating a prediction method according to the present invention.

### DESCRIPTION OF EMBODIMENTS

Hereinafter, embodiments of the present invention will be described in detail. In order to facilitate understanding of the description, the same reference numerals are given to the same components in the drawings, and redundant description will be omitted. In the present specification, “to” indicating a numerical range means that numerical values described before and after the “to” are included as a lower limit value and an upper limit value, unless otherwise specified.

### <Learning Device>

A learning device according to the present embodiment will be described. A learning device according to the present embodiment performs machine learning using a feature, such as a molecular structure of molecules of interest, as an explanatory variable and a feature indicating an intermolecular space between the molecules of interest in a simulation cell having a freely chosen parallelepiped shape as an objective variable, and generates a trained model for predicting, from the molecules of interest, a feature indicating an intermolecular space between the molecules of interest.

In the present embodiment, molecules of interest are molecules making up a base oil that is liquid at normal temperature of 20° C. and normal pressure of 1.013×10⁵ Pa.

The base oil is an oil used as a base material for a lubricant, grease, or the like, and has a function of, for example, uniformly and stably dissolving an additive and feeding the additive to a place requiring it for lubrication. The base oil is mainly classified into mineral oils obtained by petroleum refining, synthetic oils obtained by chemical synthesis, mixed oils thereof, and the like. The lubricant or grease is a mixture containing one or more base oils and one or more additives. Examples of the lubricant include engine oils, hydraulic oils, compressor oils, turbine oils, gear oils, and refrigerator oils, which are used in industrial machines such as internal combustion engines, hydraulic machines, compression machines, turbines, gear elements, bearings, and refrigerators. Examples of the additives include plasticizers, stabilizers, oiliness agents, friction modifiers, anti-wear agents, antioxidants, ultraviolet absorbers, lubricants, mold release agents, antistatic agents, rust inhibitors, defoaming agents, viscosity index improvers, and the like.

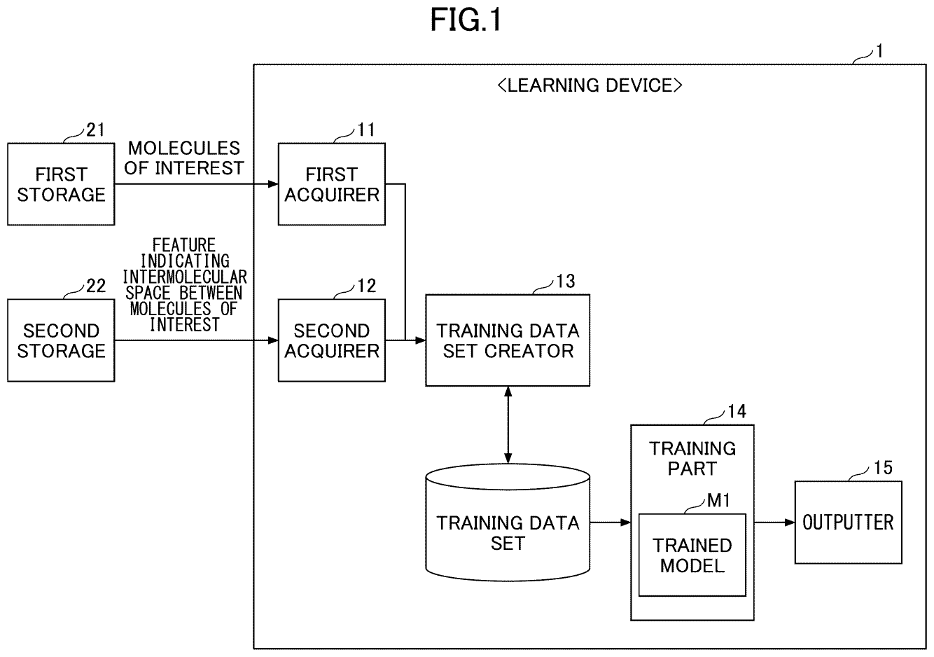

FIG. 1 is a block diagram illustrating a schematic configuration of the learning device according to the present embodiment. As illustrated in FIG. 1, the learning device 1 includes a first acquirer 11, a second acquirer 12, a training data set creator 13, a training part 14, and an outputter 15. The learning device 1 generates a trained model M1 for predicting a feature indicating an intermolecular space between molecules of interest in a simulation cell having a freely chosen parallelepiped shape.

The first acquirer 11 acquires information regarding molecules of interest, which are molecules included in a base oil, as an explanatory variable.

The information regarding molecules of interest may be acquired from the first storage 21.

The first storage 21 stores a data table including information regarding a lubricant and other information.

The information regarding a lubricant includes information regarding a type of a lubricant, a base oil and an additive included in the lubricant, and the like.

The type of a lubricant is engine oil, hydraulic oil, gear oil, refrigerator oil, or the like.

Examples of the information regarding a base oil and an additive include the types of a base oil and an additive, components and structural formulae constituting the base oil and the additive, and ratios of the base oil and the additive.

Examples of a base oil include mineral oils, synthetic oils, animal and plant oils, and mixed oils thereof.

Examples of an additive include plasticizers, stabilizers, oiliness agents, friction modifiers, anti-wear agents, metallic detergents, antioxidants, anti-friction and anti-wear agents, extreme pressure agents, ultraviolet absorbers, lubricants, mold release agents, ashless dispersants, oiliness improvers, antistatic agents, rust inhibitors, antifoaming agents, viscosity index improvers, metal deactivators, and solid lubricants.

Examples of components constituting a base oil include components constituting oils generally used as base oils, such as mineral oils, synthetic oils, animal and plant oils, and mixed oils thereof.

Examples of mineral oils include paraffin-based crude oils, naphthene-based crude oils, intermediate-based crude oils, aromatic-based crude oils, distillate oils, and refined oils. Examples of paraffin-based crude oils include isoparaffin and paraffin. Examples of naphthene-based crude oils include naphthene.

Examples of synthetic oils include poly-α-olefins, polyisobutylene (polybutene), monoesters, diesters, polyol esters, silicate esters, polyalkylene glycols, polyphenyl ethers, silicones, fluorine compounds, alkylbenzenes, and GTL base oils.

Examples of animal or vegetable oils include vegetable oils and fats such as castor oil, olive oil, cacao butter, sesame oil, rice bran oil, safflower oil, soybean oil, camellia oil, corn oil, rapeseed oil, palm oil, palm kernel oil, sunflower oil, cottonseed oil, and coconut oil, and animal oils and fats such as beef tallow, lard, milk fat, fish oil, and whale oil.

Examples of components constituting an additive include an ester compound of a monovalent or polyvalent aliphatic carboxylic acid and a monovalent or polyvalent aliphatic alcohol.

Examples of the monovalent aliphatic carboxylic acid used for the synthesis of an ester compound include saturated aliphatic carboxylic acids, such as methanoic acid, acetic acid, propionic acid, butyric acid, pentanoic acid, caproic acid, heptanoic acid, octanoic acid, nonanoic acid, decanoic acid, undecanoic acid, dodecanoic acid, tridecanoic acid, tetradecanoic acid, pentadecanoic acid, hexadecanoic acid, heptadecanoic acid, octadecanoic acid, nonadecanoic acid, icosanoic acid, heneicosanoic acid, docosanoic acid, tricosanoic acid, tetracosanoic acid, pentacosanoic acid, hexacosanoic acid, heptacosanoic acid, octacosanoic acid, nonacosanoic acid, and triacontanoic acid, etc.

Examples of the polyvalent aliphatic carboxylic acid used for the synthesis of an ester compound include saturated aliphatic carboxylic acids, such as oxalic acid, malonic acid, succinic acid, glutaric acid, adipic acid, pimelic acid, suberic acid, azelaic acid, sebacic acid, undecanedioic acid, dodecanedioic acid, tridecanedioic acid, tetradecanedioic acid, heptadecanedioic acid, and hexadecanedioic acid.

Examples of the monovalent aliphatic alcohol used for the synthesis of an ester compound include methanol, ethanol, propanol, butanol, pentanol, hexanol, heptanol, octanol, nonanol, decanol, undecanol, dodecanol, tridecanol, tetradecanol, pentadecanol, and hexadecanol.

Examples of the polyvalent aliphatic alcohol used for the synthesis of an ester compound include ethylene glycol, propylene glycol, neopentyl glycol, glycerin, trimethylolethane, trimethylolpropane, pentaerythritol, and sorbitan.

The first storage 21 may use, for example, RDKit included in a library such as Anaconda (registered trademark), which is software distributed by Anaconda Corporation of the United States. If the structural formula is written in SMILES, the first acquirer 11 reads the character string of SMILES by using the MolFromSmiles included in RDKit, and reads the structural formula of a molecule.

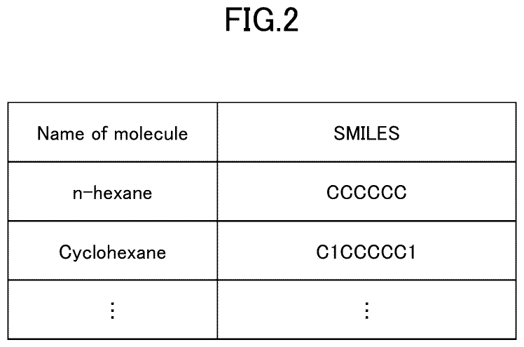

As the information regarding molecules of interest acquired from the first storage 21 by the first acquirer 11, name, structural formula, and the like of the molecules of interest may be used. As structural formula, SMILES or the like may be used. SMILES is a description of a molecular structure with a string of characters. An example of a table in which structural formulae (SMILES) are listed is illustrated in FIG. 2. As illustrated in FIG. 2, SMILES of molecules of interest are listed. The table containing the structural formulas of molecules of interest may be obtained from data in the format of CSV, spreadsheet software Excel, etc. The first acquirer 11 may input a table in which SMILES of potential molecules of interest are written as illustrated in FIG. 2.

SMILES may be obtained from a chemical database, such as PubChem (a database of chemicals provided by NCBI in the United States).

The second acquirer 12 acquires, as an objective variable, a feature indicating an intermolecular space between molecules of interest in a simulation cell having a freely chosen shape such as a parallelepiped.

The feature indicating an intermolecular space between molecules of interest may be acquired from the second storage 22.

The second storage 22 may store a database indicating a relationship between molecules of interest and a feature indicating an intermolecular space between molecules of interest in a simulation cell having a freely chosen shape.

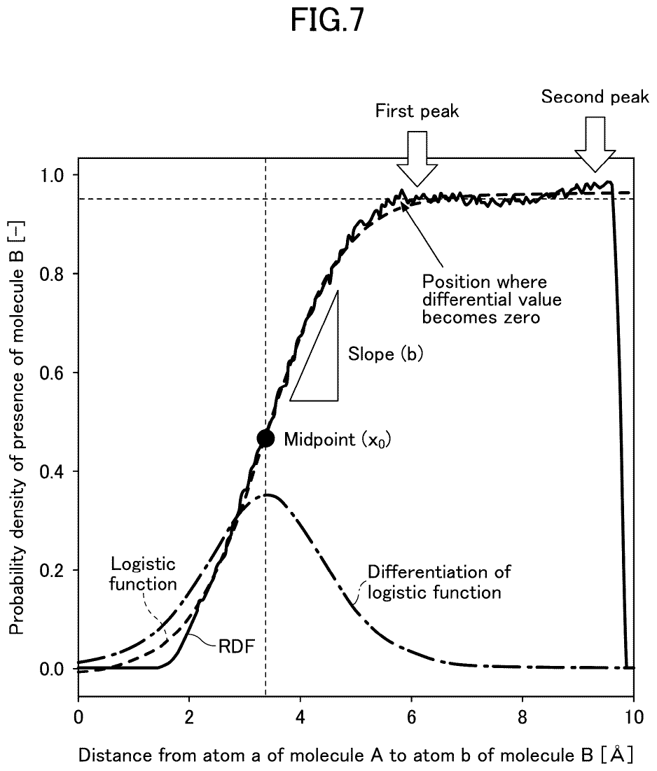

The feature indicating an intermolecular space between molecules of interest is a value calculated based on a radial distribution function representing the relationship between an atomic distance between different molecules and an abundance ratio of one molecule to another molecule in a liquid optimized structure obtained by stabilizing a liquid structure created from a three-dimensional structure of a freely chosen number of molecules of interest in a simulation cell. As a feature indicating an intermolecular space between molecules of interest, a midpoint or a slope of an approximate expression of a radial distribution function, a first peak or a second peak of the radial distribution function, or a value of the horizontal axis when a differential value of the radial distribution function approaches approximately zero may be used, as described later (see FIG. 7). The midpoint, the slope, etc. of the approximate expression of the radial distribution function correlate with an atomic distance between different molecules, and it can be said that they correlate with a degree of density of an oil film formed of a base oil composed of molecules of interest.

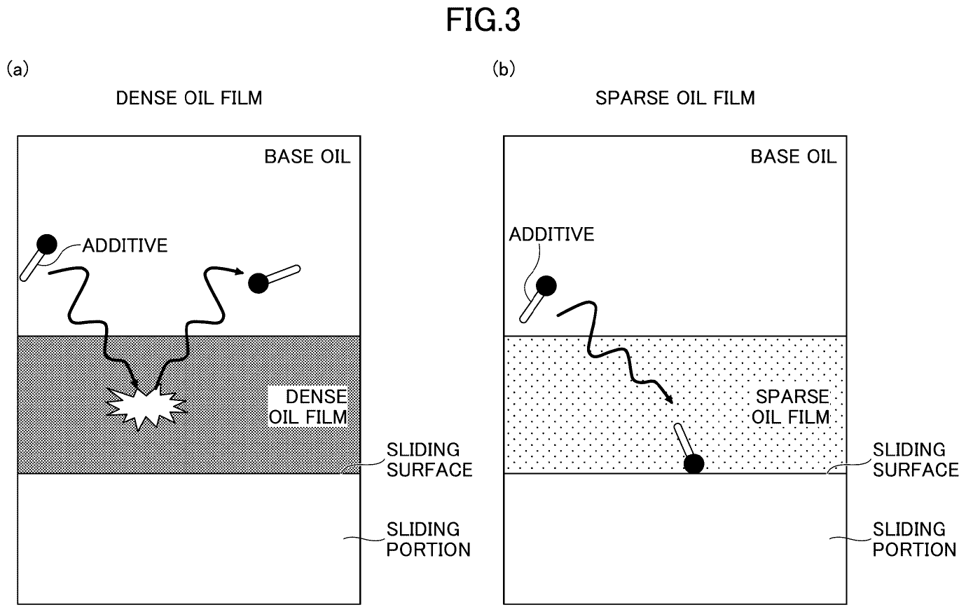

As illustrated in FIG. 3, in a sliding portion of an industrial machine or the like, with respect to a sliding surface that rubs against a surface of another sliding component (hereinafter, simply referred to as a “sliding surface of a sliding portion”), as an oil film formed of a base oil composed of molecules of interest becomes denser, an additive contained in the base oil is less likely to pass through the inside of the oil film and is less likely to reach the sliding surface; therefore, the effect of the additive tends to less likely be exhibited (see FIG. 3(a)). As the oil film becomes sparser, gaps are more likely to be generated in the oil film, and the additive is more likely to pass through the gaps in the oil film, and thus the additive migrates inside the oil film and easily reaches the sliding surface (see FIG. 3(b)). Therefore, the effect of the additive, such as friction reduction, tends to be easily exhibited. Therefore, if a feature correlated with an atomic distance between atoms of different molecules (for example, a slope, a midpoint, or the like of an approximate expression of a radial distribution function, which will be described later), instead of an atomic distance in one molecule, is used as a feature of molecules of interest included in a base oil, a degree of density of the oil film formed from a feature indicating an intermolecular space between molecules of interest can be predicted.

A feature indicating an intermolecular space between molecules of interest may be calculated by performing a molecular simulation such as a molecular dynamics method using an optimal liquid structure of a single-molecule structure of molecules of interest included in a base oil.

A feature indicating an intermolecular space between molecules of interest may be, for example, a value calculated by a molecular feature calculation device.

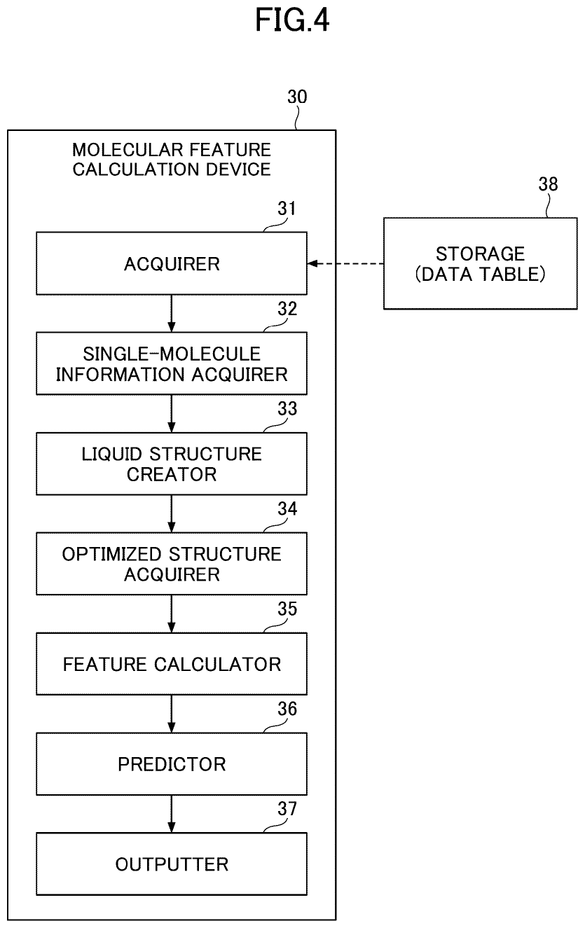

The molecular feature calculation device is not particularly limited as long as a device can calculate a feature indicating an intermolecular space between molecules of interest through performing a molecule simulation using a single-molecule structure of molecules of interest included in a base oil. An example of a functional block diagram illustrating the configuration of a molecular feature calculation device is shown in FIG. 4. As illustrated in FIG. 4, a molecular feature calculation device 30 may include an acquirer 31, a single-molecule information acquirer 32, a liquid structure creator 33, an optimized structure acquirer 34, a feature calculator 35, a predictor 36, and an outputter 37.

The acquirer 31 acquires, from the storage 38, information regarding molecules of interest that are molecules included in a base oil, and acquires information regarding an additive.

The storage 38 stores a data table including information regarding molecules of interest and additives. The information regarding molecules of interest and additives may be the same as the information stored in the first storage 21 and the second storage 22. Since the information regarding molecules of interest and additives may be the same as the information stored in the first storage 21 and the second storage 22, the details of the information are omitted.

As information regarding molecules of interest, name and structural formula of the molecules of interest may be used. As structural formula, SMILES or the like may be used. Since SMILES is the same as the above, the details are omitted.

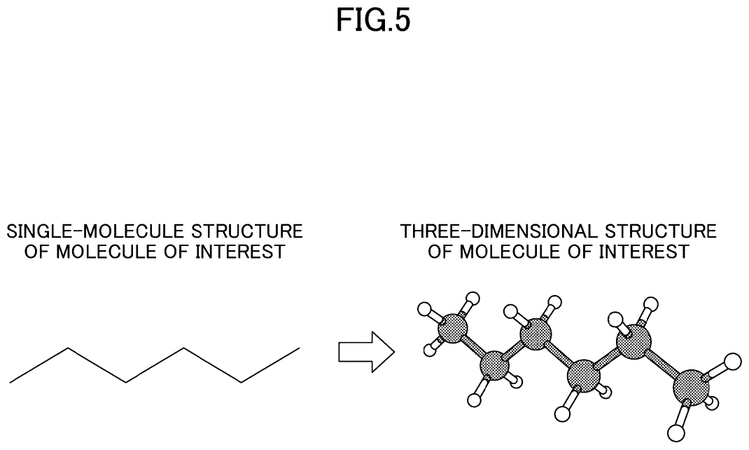

The single-molecule information acquirer 32 acquires a three-dimensional structure of a molecule of interest acquired by the acquirer 31 as single-molecule information. For example, as illustrated in FIG. 5, the single-molecule information acquirer 32 adds the hydrogens to the single-molecule structure of the molecule of interest to create a three-dimensional structure of the molecule of interest in which the hydrogens are added to the single-molecule structure of the molecule of interest, and acquires the three-dimensional structure as single-molecule information.

The single-molecule information acquirer 32 may acquire an optimized structure in which the three-dimensional structure is optimized, as single-molecule information. The single-molecule information acquirer 32 may arrange the atoms constituting a molecule of interest at appropriate positions in consideration of the relationship between the coordinates and the energy of the atoms, and obtain a structure in which the three-dimensional structure is most stable in terms of energy. The single-molecule information acquirer 32 may create an optimized structure using a general molecular simulation method, such as first-principles calculations or molecular mechanics calculations.

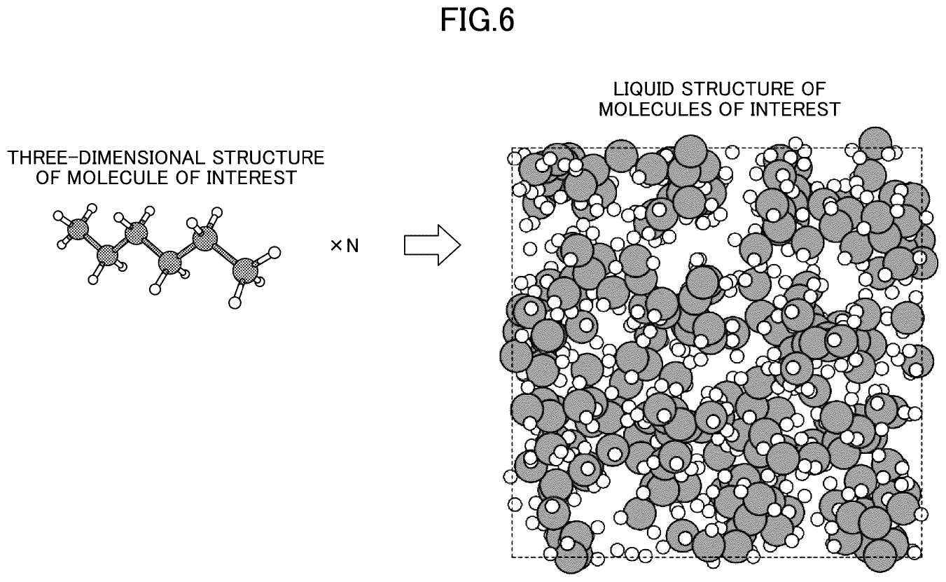

The liquid structure creator 33 creates a liquid structure by arranging a freely determined number (N) of units of single-molecule information acquired by the single-molecule information acquirer 32 inside a simulation cell as illustrated in FIG. 6. The number of units of single-molecule information is not particularly limited and may be set as appropriate, but may be set to several tens to several hundreds. The simulation cell may be defined to have any freely chosen shape such as a parallelepiped. The size of a simulation cell is set freely as appropriate according to the type of a lubricant or molecules of interest, or the like. The simulation cell may be defined, for example, to be approximately the same as the actual density of the base oil, so that the actual structure of the base oil is reproduced.

The liquid structure creator 33 arranges a freely determined number (N) of units of single-molecule information in a simulation cell at different positions so that the N units of single-molecule information do not overlap. The liquid structure creator 33 may create a liquid structure by randomly arranging and rearranging a freely determined number (N) of units of single-molecule information in a simulation cell.

As illustrated in FIG. 4, the optimized structure acquirer 34 may acquire a liquid optimized structure optimized by relaxing the liquid structure created by the liquid structure creator 33. Relaxation is a general structural optimization method, such as a steepest descent method or a conjugate gradient method, for acquiring a minimum value of energy in a multi-dimensional space. For example, relaxation means that a sum or scalar of force vectors, or a stress tensor or the main component of the stress tensor, or the like acting on the entire liquid structure is made to coincide with a predetermined pressure (external pressure), which is described later, under a certain threshold value. There may be a case where the liquid structure created by the liquid structure creator 33 does not have a correct density corresponding to an actual state because the single-molecule information is put in a simulation cell having an arbitrary size. In this state, even if a feature is calculated by the feature calculator 35, the accuracy of the calculated feature may be low. The optimized structure acquirer 34 can create a liquid optimized structure adjusted to a liquid structure having a correct density by optimizing the liquid structure.

When optimizing the liquid structure, the optimized structure acquirer 34 may create the liquid optimized structure under the following two conditions.
(1) The volume of the simulation cell is variable. An external force may be applied to the simulation cell, and the liquid structure may be in a state where a predetermined pressure (external pressure) is applied.
(2) The optimized structure acquirer 34 may create a structure under the condition (1) as a structure at the absolute zero temperature. A molecular dynamics simulation may be performed to take the effects of temperature into consideration. Examples of the molecular dynamics simulation include structural optimization (a molecular mechanics method), a molecular dynamics method, and a Monte Carlo method. The volume of the simulation cell may be variable. The temperature may be set to any value as appropriate. An external force may be applied to the simulation cell, and the liquid structure may be in a state where a predetermined pressure (external pressure) is applied.

The liquid optimized structure is created under the above-described two conditions. The simulation cell is thereby defined to reproduce a density of the actual base oil. In other words, the simulation cell is defined in such a manner that the liquid structure has a density when relaxed in consideration of a predetermined pressure and temperature.

The temperature and pressure may be high, for example, when a lubricant in a contact state is simulated. The high temperature may be, for example, 50° C. to 200° C., and specifically, about 60° C. The high pressure may be, for example, 50 MPa to 2000 MPa, and specifically, about 500 MPa. The high pressure may be, for example, 50 MPa to 2000 MPa, and specifically, about 500 MPa.

The optimized structure acquirer 34 may create a liquid optimized structure using a general molecular simulation method, such as first-principles calculations or molecular mechanics calculations.

The feature calculator 35 calculates a feature indicating an intermolecular space between molecules of interest by calculating an atomic distance between atoms of different molecules from a liquid optimized structure and calculating a radial distribution function (RDF) representing an abundance ratio of one molecule to another molecule at each atomic distance. The feature calculator 35 may use a feature represented in a radial distribution function (for example, a slope, a midpoint, or the like of an approximate expression of a radial distribution function, which will be described later) represented based on an atomic distance between atoms of different molecules, instead of an atomic distance in one molecule.

An atomic distance between atoms of different molecules is a distance between two atoms included in two molecules respectively. Hereinafter, one molecule is referred to as “molecule A”, and another molecule is referred to as “molecule B”. An atomic distance between atoms of different molecules may be a distance between “atom a” included in molecule A and an atom included in molecule B existing at a position corresponding to atom a, or may be a distance between atom a included in molecule A and an atom included in molecule B existing at a position corresponding to an atom other than atom a.

The feature calculator 35 calculates an atomic distance between atoms of different molecules from a liquid optimized structure acquired by the optimized structure acquirer 34, and calculates a radial distribution function representing an abundance ratio of one molecule to another molecule at each atomic distance. The radial distribution function may be calculated, for example, by the following Expression (1).

$$\begin{matrix}[ Expression 1 ] & \\ {g}_{A - B} ( r ) = \frac{〈 {N}_{A - B} 〉}{4 π {r}^{2} \cdot Δ r \cdot {ρ}_{B}} & ( 1 )\end{matrix}$$

(wherein A is one molecule, B is another molecule different from molecule A, r is a radial distance as viewed from atom a of molecule A, g_{A-B}(r) is a probability of presence of atom b of molecule B existing at a radial distance r as viewed from atom a of molecule A, <N_{A-B}> is an ensemble average of the number of atom b of molecule B existing in a section ranging from r−½Δr to r+½Δr as viewed from atom a of molecule A, Δr is a spherical section, and ρ_{B} is a number density of B.)

FIG. 7 shows an example of a radial distribution function illustrating a relationship between atom a of molecule A and atom b of molecule B. A radial distribution function is obtained in which a probability density of presence of molecule B rises from a constant state of 0 toward 1 as a distance from atom a of molecule A to atom b of molecule B increases as illustrated in FIG. 7, and the vertical axis value becomes substantially constant near 0.95 for a horizontal axis section illustrated in FIG. 7.

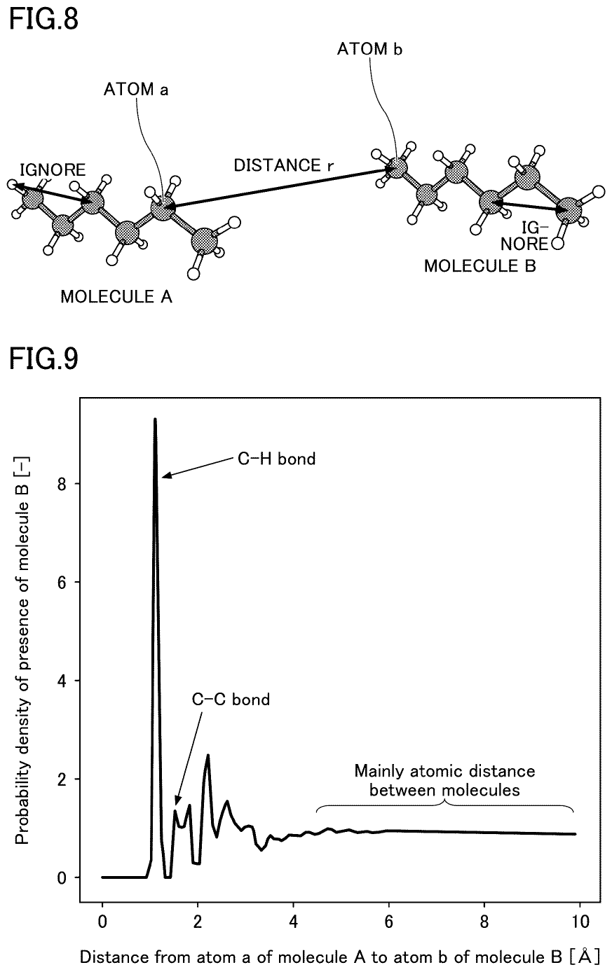

A radial distribution function indicates a probability that atom b of molecule B exists at a distance r from atom a of molecule A, as illustrated in FIG. 8. In other words, a radial distribution function represents a frequency of atom b of molecule B being positioned at a distance r from atom a of molecule A, and correlates with an intermolecular distance between atom a of molecule A and atom b of molecule B. A radial distribution function may be plotted in multiple dimensions, instead of two dimensions.

The feature calculator 35 may convert the calculated radial distribution function into an approximate expression. The feature calculator 35 may obtain an approximate expression of the radial distribution function by fitting the radial distribution function with a logistic function of the following Expression (2) or the like.

$$\begin{matrix}[ Expression 2 ] & \\ y = \frac{a}{1 + {e}^{- b ( x - {x}_{0} )}} + c & ( 2 )\end{matrix}$$

(wherein y is a probability density of presence of molecule B, a and c are coefficients, b corresponds to a slope of the approximate expression, x is a distance (A) from an atom in molecule A to an atom in molecule B, and x₀ is the position of the midpoint of the slope of the approximate expression.)

The feature calculator 35 may calculate a feature indicating an intermolecular space between molecules of interest by extracting a parameter of the approximate expression into which the radial distribution function is converted. The parameter may be the midpoint or the slope of the approximate expression of the radial distribution function. In other words, in the case where the approximate expression of the radial distribution function is obtained using the logistic function of the above Expression (2), the position of the midpoint of the approximate expression of the radial distribution function is used as the parameter x₀ and the slope of the approximate expression is used as the parameter b in the logistic function of Expression (2). Therefore, as illustrated in FIG. 7, the midpoint or the slope of the approximate expression of the radial distribution function can be used as a parameter of a feature indicating an intermolecular space, of molecules of interest, and the midpoint or the slope of the approximate expression of the radial distribution function is correlated with the atomic distance between atoms of different molecules and is correlated with the degree of density of the oil film.

A midpoint and a slope of an approximate expression correspond to a distance between molecules. The larger the midpoint or the smaller a slope, the more molecules are separated from each other, which means that a relatively sparse oil film is formed, and the adsorption time until an additive is adsorbed on the surface is short. The smaller a midpoint of the approximate expression or the larger a slope, the closer molecules are, which means that a relatively dense oil film is formed, and the adsorption time until an additive is adsorbed on the surface is long. Therefore, the adsorption time of an additive becomes shorter as a midpoint of an approximate expression becomes larger or a slope becomes smaller. If an additive can be adsorbed on a sliding surface of a sliding part of an industrial machine or the like in a shorter time, an effect of the additive can be exhibited, and if an additive can be adsorbed by a sliding surface of a sliding part, an effect of the additive can be exhibited and the surface of a target can be protected from severe friction; therefore, effects such as low friction and low wear can be obtained. Therefore, it is preferable to adjust an oil film to have a sparse structure by increasing a midpoint of an approximate expression or decreasing a slope to increase an intermolecular space.

As illustrated in FIG. 7, the feature calculator 35 may use, as a feature indicating an intermolecular space between molecules of interest, a first peak (a local maximum value that appears first when viewed from a short distance to a long distance), a second peak (a local maximum value that appears second when viewed from a short distance to a long distance), or a value on the horizontal axis when a differential value of the radial distribution function asymptotically approaches substantially zero, of the radial distribution function.

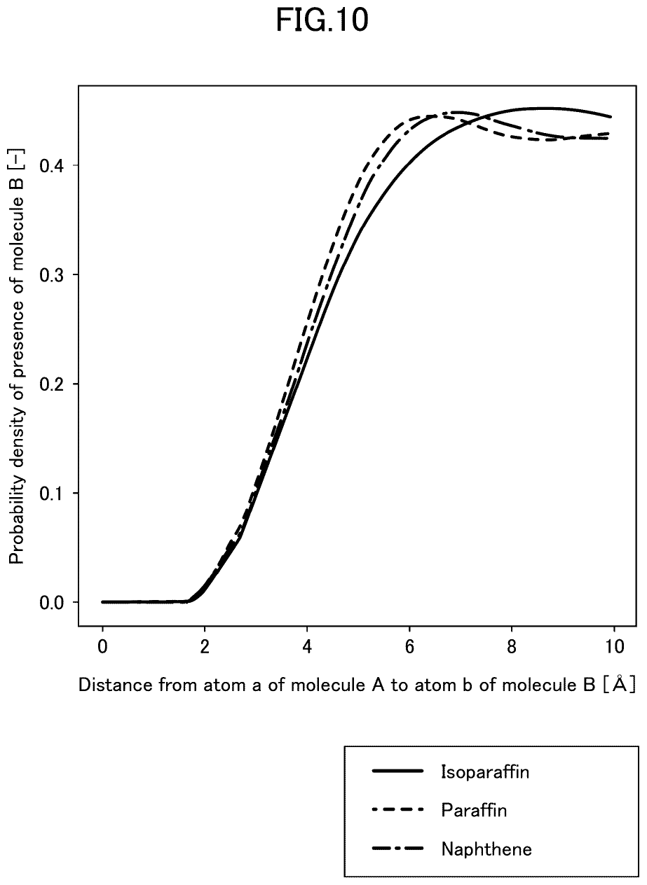

The feature calculator 35 uses a feature correlated with an atomic distance between different molecules (for example, a slope, a midpoint, or the like of an approximate expression of a radial distribution function), as a feature indicating an intermolecular space between molecules of interest included in the base oil. The feature calculator 35 can specify an intermolecular space by obtaining a radial distribution function focusing only on an atomic distance between molecules, because completely different features appear depending on a type of molecules. For example, in the case of a radial distribution function of a common normal alkane liquid, as illustrated in FIG. 9, since many C—H bonds and C—C bonds are present in one molecule, sharp peaks derived from the C—H bonds and the C—C bonds are present in an area where the distance between atoms in one molecule is short. In an area where the distance between molecules is long, a peak or the like relating to an intermolecular pair is present, but such a peak is relatively weak as compared with the atomic distance in one molecule, and is hardly recognized. If the atomic distance in one molecule such as a C—H bond or a C—C bond is excluded, as illustrated in FIG. 10, a radial distribution function in which only the distance between atoms of different molecules appears is obtained. A radial distribution function that represents only the distance between atoms of different molecules, such as isoparaffin, paraffin, and naphthene, has a completely different shape depending on the type of molecule, and thus is effective in specifying a space between atoms of different molecules.

The feature calculator 35 may use various physical properties, such as viscosity, flash point, diffusion coefficient, and thermal conductivity of the base oil, in addition to the midpoint or the slope of the approximate expression of the radial distribution function, as a feature indicating an intermolecular space between molecules of interest.

The predictor 36 predicts a degree of density of an oil film formed of molecules of interest based on a magnitude of a feature indicating an intermolecular space between the molecules of interest, such as the midpoint or the slope extracted from the approximate expression of the radial distribution function indicating an intermolecular space between molecules, which is calculated by the feature calculator 35. Specifically, the predictor 36 predicts a degree of density of an oil film formed of molecules of interest by measuring whether the feature of the molecules of interest calculated by the feature calculator 35 is larger or smaller than a feature of a base oil currently used.

For example, in the case where the feature indicating an intermolecular space between molecules of interest is the midpoint extracted from the approximate expression of the radial distribution function, if the feature calculated by the feature calculator 35 is larger than the feature of the base oil currently used (reference feature), the predictor 36 predicts that the oil film is sparse, and predicts a degree of sparsity of the oil film according to the magnitude of the feature. When the feature calculated by the feature calculator 35 is smaller than the feature of the base oil currently used (reference feature value), the predictor 36 predicts that the oil film is dense, and predicts a degree of density of the oil film according to the smallness of the feature.

The outputter 37 outputs the prediction result of the density of the oil film formed of the base oil composed of the molecules of interest inside the simulation cell, which is predicted by the predictor 36, through display, transmission, or the like.

As illustrated in FIG. 1, the training data set creator 13 extracts molecules of interest as an explanatory variable and a feature indicating an intermolecular space between molecules of interest in a simulation cell having a freely chosen parallelepiped shape as an objective variable, and adds these variables to the training data set. The training data set creator 13 creates a training data set by linking the input molecules of interest and the input feature indicating an intermolecular space between the molecules of interest to each other.

The training part 14 generates a trained model M1 through training using a training data set in which molecules of interest (an explanatory variable) and a feature indicating an intermolecular space between the molecules of interest in a simulation cell having a freely chosen parallelepiped shape (an objective variable) are associated with each other.

The trained model M1 is a trained model for which machine learning has been performed in advance using a training data set (training data table) stored in a storage (not illustrated), and the training result of the correspondence relationship between molecules of interest (an explanatory variable) and a feature indicating an intermolecular space between molecules of interest in a simulation cell having a freely chosen parallelepiped shape (an objective variable), which are obtained by performing machine learning using the training data set stored in the storage, is applied. The trained model M1 is a program for modeling the input-output relationship between molecules of interest and a feature indicating an intermolecular space between molecules of interest, wherein molecules of interest is input data as an explanatory variable and a feature indicating an intermolecular space between molecules is output data as an objective variable. The trained model M1 may be represented by a mathematical expression such as a function.

It is preferable that the trained model M1 apply a supervised learning algorithm among machine learning algorithms. Examples of the supervised learning include linear regression, regularized regression, partial least squares regression, polynomial regression, kernel regression, logistic regression, random forest, gradient boosting regression tree, support vector machine (SVM), neural network, and so on. The neural network can use deep learning, which is a neural network with more layers than three layers. Examples of the type of the neural network include a convolutional neural network (CNN), a recurrent neural network (RNN), a general regression neural network, and so on. Among these, it is preferable to use a gradient boosting regression tree.

The outputter 15 displays information on a training data set used in training of the trained model M1, information on the trained model M1, and the like.

As described above, the learning device 1 provided with the training part 14 can generate the trained model M1 that predicts a feature indicating an intermolecular space between molecules of interest. The trained model M1 generated by the learning device 1 predicts a feature indicating an intermolecular space between molecules of interest from information regarding molecules of interest that are input.

The feature indicating an intermolecular space between molecules of interest correlates with an atomic distance between atoms of different molecules and correlates with a degree density of an oil film formed of a base oil composed of molecules of interest. The sparser an oil film, the more easily an additive passes through the gaps in the oil film, and the more easily the additive reaches a sliding surface of a sliding part of an industrial machine; therefore, an effect of the additive, such as the reduction of friction, is easily exhibited. Thus, the learning device 1 generates the trained model M1 to be used for prediction of a degree of density of an oil film formed of a base oil composed of molecules of interest that are input; therefore, it is possible to shorten time required for selecting a base oil that brings out an effect of an additive.

For example, in a case where a degree of density of an oil film formed of a base oil composed of molecules of interest is predicted using a diffusion/adsorption simulation, that has been generally used in the related art, it takes a long time (for example, about several days to several months) to calculate a feature from a radial distribution function, even for one type of base oil, and thus it takes a longer time to predict a density of an oil film. With a use of the learning device 1, the trained model M1 can calculate a feature of one type of base oil, for example, for a short time of a few seconds to a few minutes; therefore, it is possible to predict a degree of density of an oil film more easily in a shorter time.

Since the learning device 1 can generate the trained model M1, the use of the trained model M1 can reduce the burden and time required for predicting a feature indicating an intermolecular space between molecules of interest from information regarding molecules of interest. In production of a lubricant, in order to produce a lubricant that satisfies desired performance according to the type and use of the lubricant, in practice, a lubricant is produced by combining various base oils and additives in an experiment, and verification, etc. of a degree of density is performed. Since a type of a base oil that brings out an effect of an additive, a composition of a base oil and an additive, and the like are determined by performing these steps, a lot of labor is required, and the cost burden is large due to the preparation of various base oils and additives. The learning device 1 can predict a feature indicating an intermolecular space between molecules of interest from molecules of interest, and therefore can efficiently predict a feature indicating an intermolecular space between molecules of interest while reducing the burden. Therefore, the learning device 1 can reduce the burden of predicting a degree of density of an oil film composed of molecules of interest by using a feature indicating an intermolecular space between molecules of interest for predicting a degree of density of an oil film formed of a base oil composed of molecules of interest.

The learning device 1 can use, as a feature, a value calculated based on a radial distribution function representing the relationship between an atomic distance between atoms of different molecules and an abundance ratio of one molecule to another molecule, of a liquid optimized structure obtained by stabilizing a liquid structure created from a freely chosen number of three-dimensional structures of molecules of interest within a simulation cell. Thus, the learning device 1 can be used to predict a feature indicating an intermolecular space between molecules of interest with higher accuracy. Therefore, the learning device 1 can improve the accuracy in selecting a base oil that brings out an effect of an additive.

### <Prediction Device>

The prediction device according to the present embodiment will be described. The prediction device according to the present embodiment predicts a degree of density of an oil film including molecules of interest.

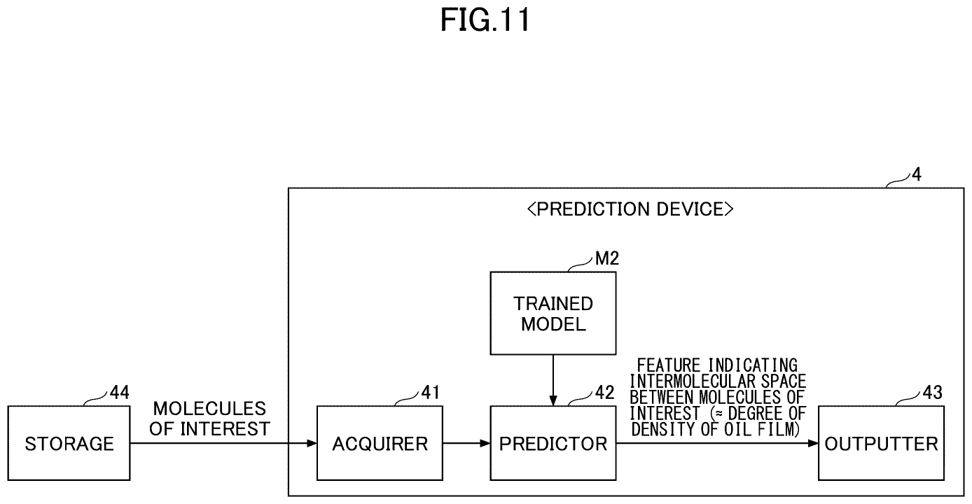

FIG. 11 is a system configuration diagram illustrating the prediction device according to the present embodiment. As illustrated in FIG. 11, the prediction device 4 includes an acquirer 41, a trained model M2, a predictor 42, and an outputter 43, and predicts, from the molecules of interest, a feature indicating an intermolecular space between molecules of interest as an index indicating a degree of density of an oil film containing the molecules of interest.

The acquirer 41 acquires molecules of interest included in a base oil, which are a prediction target, as an explanatory variable. The information regarding molecules of interest may be acquired from the storage 44. The storage 44 stores a data table including information regarding lubricants and the like, as in the first storage 21 described above. The information regarding molecules of interest is the same as the information regarding molecules of interest acquired from the first storage 21 in the learning device 1, and therefore, the description thereof is omitted.

The trained model M2 is trained using a training data set prepared in advance, in which molecules of interest (an explanatory variable) and a feature indicating an intermolecular space between molecules of interest in a simulation cell having a freely chosen parallelepiped shape (an objective variable) are associated with each other. The trained model M1 generated by the learning device 1 can be used as the trained model M2.

The predictor 42 predicts a degree of density of an oil film formed of molecules of interest by inputting molecules of interest as a prediction target, which is acquired by the acquirer 41, using the trained model M2. In other words, the predictor 42 inputs the molecules of interest as a prediction target, which is acquired by the acquirer 41, to the trained model M2, and outputs a feature indicating an intermolecular space between the molecules of interest as a prediction target, which is predicted by the trained model M2, as an objective variable. The objective variable is output by the predictor 42 as an index indicating a degree of density of an oil film formed of a base oil composed of the molecules of interest as a prediction target. As described above, the feature indicating an intermolecular space between molecules of interest correlates with an atomic distance between atoms of different molecules and correlates with a degree of density of an oil film formed of a base oil composed of molecules of interest. Therefore, the predictor 42 can predict, from the trained model M2, a degree of density of an oil film formed of a base oil composed of molecules of interest as a prediction target by predicting a feature indicating an intermolecular space between molecules of interest corresponding to the molecules of interest as a prediction target.

The outputter 43 outputs a feature indicating an intermolecular space between molecules of interest predicted by the trained model M2 as an objective variable, and outputs, in a form of display or the like, the feature as a value indicating a degree of density of an oil film formed of a base oil composed of molecules of interest.

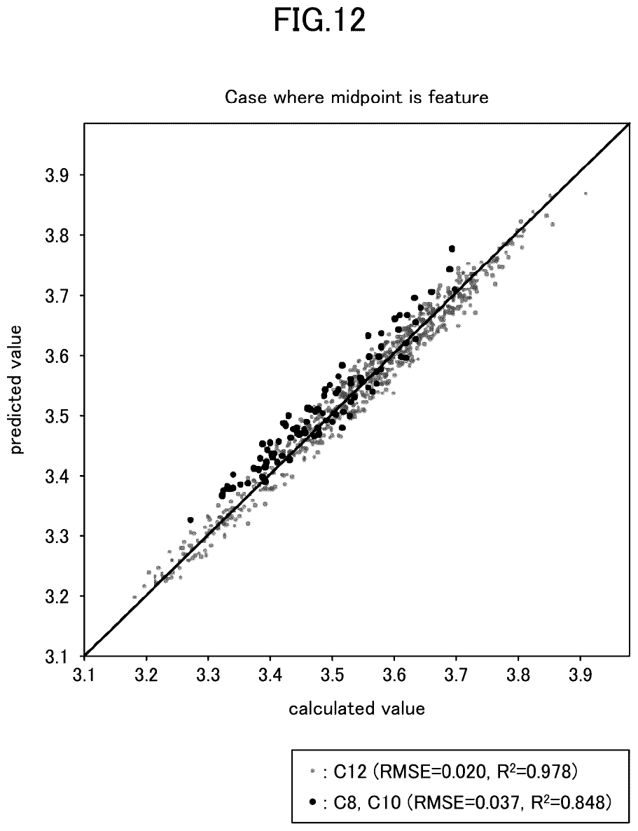

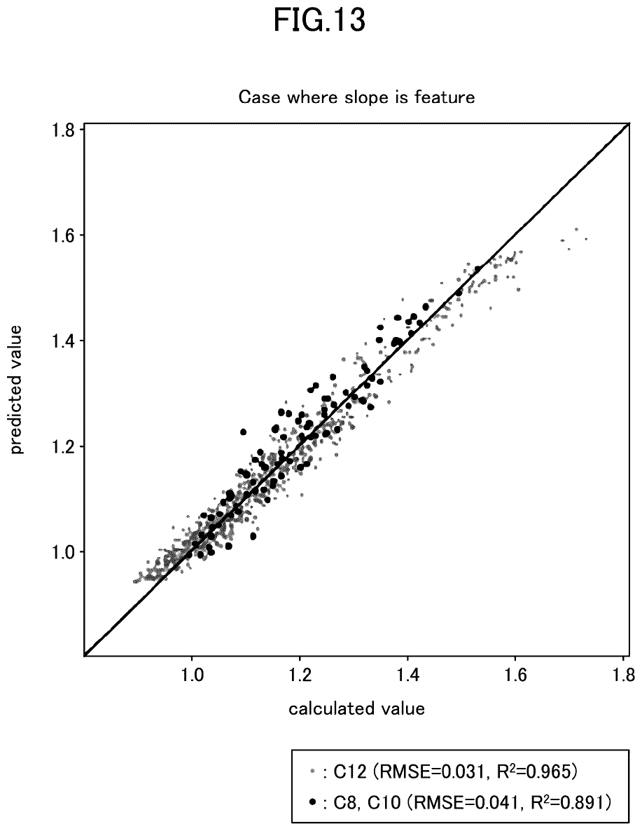

FIGS. 12 and 13 illustrate an example of the accuracy of a prediction value predicted by the predictor 42 using the trained model M2. FIG. 12 illustrates a case where a feature is a midpoint of an approximate expression (see FIG. 7) of a radial distribution function, and FIG. 13 illustrates a case where a feature is a slope of an approximate expression (see FIG. 7) of a radial distribution function. FIGS. 12 and 13 illustrate a case where n-octane (C₈H₁₈ (hereinafter, also simply referred to as “C8”)), n-decane (C₁₀H₂₂ (hereinafter, also simply referred to as “C10”)), and n-dodecane (C₁₂H₂₆ (hereinafter, also simply referred to as “C12”)) are used as the linear saturated hydrocarbon. The trained model M2 is generated using the training data consisting of only C12.

FIG. 12 is a graph in which the relationship between calculated values obtained by actually calculating features (a midpoint of an approximate expression of a radial distribution function) of C8, C10, and C12 and predicted values obtained by predicting features using the trained model M2 is plotted. The calculated values were calculated by using the molecular feature calculation device 30 illustrated in FIG. 4.

As illustrated in FIG. 12, in the case where a feature is a midpoint of an approximation expression, a root mean square error (RMSE) of a line representing the relationship between the plotted calculated and predicted values of a plurality of plotted C12 is 0.020, and the coefficient of determination (R²) is 0.978. An RMSE of the line representing the relationship between the plotted calculated and predicted values of a plurality of C8 and C10 plotted is 0.037, and R² is 0.848.

FIG. 13 is a graph in which the relationship between calculated values obtained by actually calculating features (a slope of an approximate expression of a radial distribution function) of C8, C10, and C12 and predicted values obtained by predicting features using the trained model M2 is plotted. The calculated values were calculated by using the molecular feature calculation device 30 illustrated in FIG. 4, as in FIG. 12.

As illustrated in FIG. 13, when a feature is a slope of an approximation expression, an RMSE of a line representing the relationship between the plotted calculated and predicted values of a plurality of C12 is 0.031, and R² is 0.965. An RMSE of the line representing the relationship between the plotted calculated and predicted values of a plurality of C8 and C10 plotted is 0.041, and R² is 0.891.

Therefore, in the case where a feature of a radial distribution function is a midpoint and a slope of an approximate expression, the predicted values predicted by the trained model M2 can be predicted with high accuracy, since an RMSE is on the order of 1/100 of the numerical values of the midpoint and the slope, which is sufficiently small, and R² is close to 1. It can be said that a feature of a radial distribution function can be predicted well even with unlearned data such as C8 and C10.

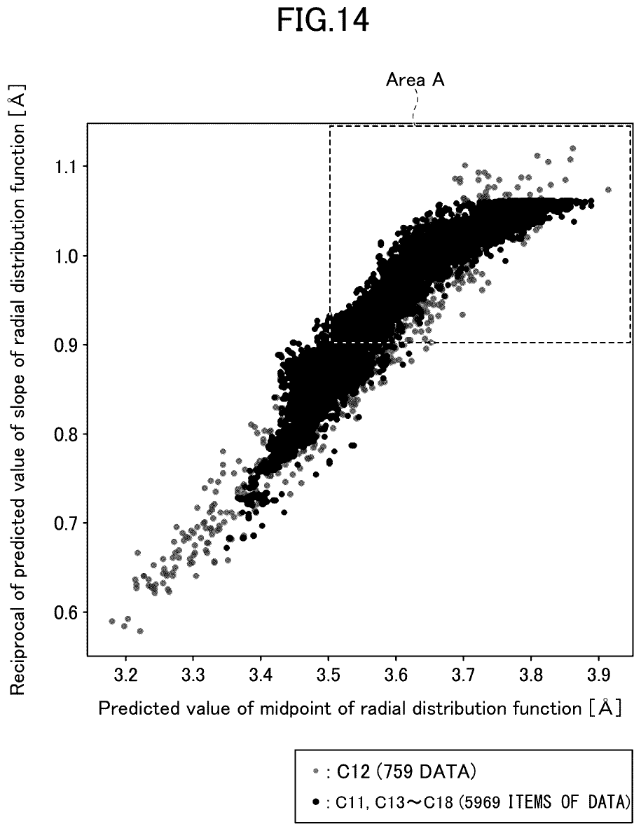

An example of the relationship between the predicted values of two features is illustrated in FIG. 14. FIG. 14 illustrates a case where the features are a predicted value of a midpoint of an approximate expression of a radial distribution function illustrated in FIG. 12 and a reciprocal of a predicted value of a slope of an approximate expression of a radial distribution function illustrated in FIG. 13. In FIG. 14, n-undecane (C₁₁H₂₄ (hereinafter, also simply referred to as “C11”)), n-tridecane (C₁₃H₂₈ (hereinafter, also simply referred to as “C13”)), and ~n-octadecane (C₁₈H₃₈ (hereinafter, also simply referred to as “C18”)) are used as the linear saturated hydrocarbon.

As illustrated in FIG. 14, it can be regarded from the plotting the relationship between the predicted values of the two features that, for example, molecules present in the area A indicated by the broken line have a structure in which the oil film formed when the molecules are used as the base oil is sparse, and brings out an effect of an additive. Therefore, it can be judged that the molecules plotted in the area A are preferable as candidate molecules to be used as the base oil.

As described above, the prediction device 4 includes the predictor 42, and the predictor 42 can predict a feature indicating an intermolecular space between molecules of interest from molecules of interest by the trained model M2, and can predict a degree of density of an oil film. The prediction device 4 can predict a degree of passage of an additive through an oil film by predicting a degree of density of an oil film by the predictor 42. In the case where an atomic distance between molecules is large in an oil film formed of a base oil composed of molecules of interest, gaps are likely to be generated. As an oil film is sparser, an additive can migrate through the gaps in the oil film, and therefore, the additive can be quickly adsorbed to a sliding surface of a sliding portion of an industrial machine or the like with which the oil comes into contact, and the effect of the additive can be easily exhibited.

In a case where a degree of density of an oil film formed of a base oil composed of molecules of interest is predicted using a diffusion/adsorption simulation, that has been generally used in the related art, it takes a long time (for example, about several days to several months) to calculate a feature from a radial distribution function, even for one type of base oil, and thus it takes a longer time to predict a density of an oil film. The prediction device 4 can calculate a feature of one type of base oil in a short time of, for example, about several seconds to several minutes, and thus can predict a degree of density of an oil film in a shorter time and a simpler manner.

Therefore, the prediction device 4 can predict, in a short time, a feature indicating an intermolecular space between molecules of interest that is obtained from molecules of interest that are input as an index indicating a degree of density of an oil film, and therefore, time required for selecting a base oil which brings out an effect of an additive can be shortened.

The prediction device 4 provided with the predictor 42 can reduce the burden and time required for predicting a feature indicating an intermolecular space between molecules of interest. Therefore, the prediction device 4 can efficiently and simply predict a degree of density of an oil film formed of a base oil composed of molecules of interest, and therefore, the burden in predicting a degree of density of an oil film formed of a base oil composed of molecules of interest can be reduced.

The prediction device 4 can be used when selecting a base oil that brings out an effect of an additive, and thus can be suitably used for the production of a lubricant or the like. Since lubricants used in industrial machines and the like are used under severe conditions such as high pressure, high speed, high load, and high temperature, it is important to select a lubricant that can exhibit desired lubricating performance even under such environments. The prediction device 4 is used when selecting a base oil that brings out an effect of an additive, and thus it is possible to appropriately select a lubricant in accordance with the use of various lubricants used for industrial machines and the like.

The learning device 1 and the prediction device 4 may be configured as a learning system and a prediction system. In other words, although the learning device 1 is a single device such as a PC (personal computer) having various components in the device, one or more of the components may be respectively disposed outside the device and connected to each other via a network.

For example, the training data set may be provided on the cloud. In this case, the learning device 1 is configured as a learning system by a training data set connected via a network.

Similarly, the prediction device 4 may also be connected via a network, with one or more of each configuration being located outside the device.

### <Hardware Configurations of Learning Device and Prediction Device>

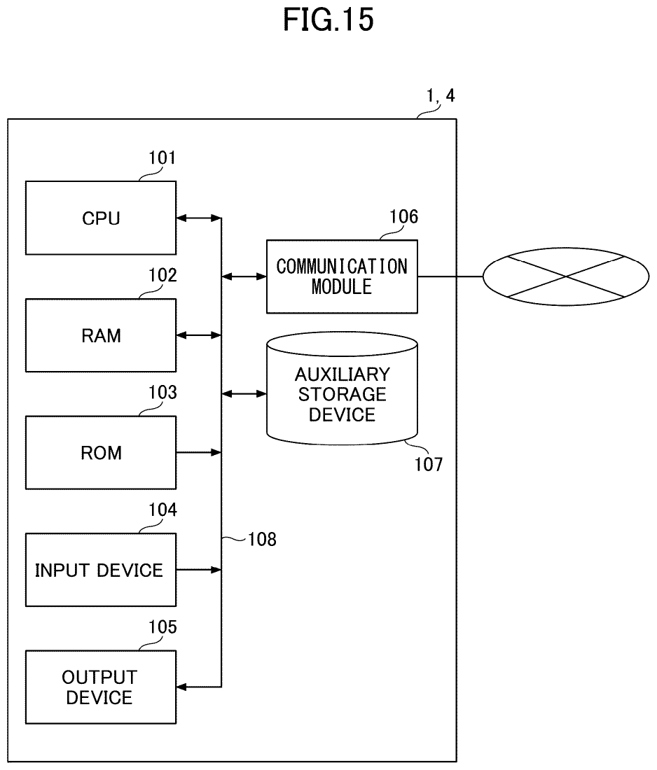

Next, an example of a hardware configuration of the learning device 1 and the prediction device 4 will be described. FIG. 15 is a block diagram illustrating a hardware configuration of the learning device 1 and the prediction device 4. As illustrated in FIG. 15, the learning device 1 and the prediction device 4 is configured by an information processing device (computer), and can be physically configured as a computer system including a central processing unit (CPU; a processor) 101 that is an arithmetic processing unit, a random access memory (RAM) 102 and a read only memory (ROM) 103 that are main storage devices, an input device 104 that is an input device, an output device 105, a communication module 106, an auxiliary storage device 107 such as a hard disk, and the like. These are connected to each other via a bus 108. The output device 105 and the auxiliary storage device 107 may be provided externally.

The CPU 101 controls the overall operation of the learning device 1 and the prediction device 4 and performs various types of information processing. The CPU 101 can learn and predict a density of an oil film formed of a base oil composed of molecules of interest by executing, for example, a training method and a prediction method or a training program and a prediction program, which will be described later, stored in the ROM 103 or the auxiliary storage device 107.

The RAM 102 may include a non-volatile RAM that is used as a work area of the CPU 101 and stores main control parameters and information.

The ROM 103 stores a basic input/output program and the like. The training program and the prediction program may be stored in the ROM 103.

The input device 104 is an input device, such as a keyboard, a mouse, an operation button, a touch panel, or a display screen, receives information input by a user as an instruction signal, and outputs the instruction signal to the CPU 101.

The output device 105 is a display device such as a monitor display, a speaker, a printing device such as a printer, or the like. In the output device 105, for example, information, such as training results and the prediction result of the density of the oil film, is displayed on a display device such as a monitor display, and a screen to be displayed is updated according to an input operation via the input device 104 or the communication module 106.

The communication module 106 is a data transmission/reception device such as a network card, and functions as a communication interface that takes in information from an external data recording server or the like and outputs analysis information to another electronic device.

The auxiliary storage device 107 is a storage device such as a solid state drive (SSD) and a hard disk drive (HDD), and stores, for example, various data, files, and the like necessary for the operation of the learning device 1 and the prediction device 4.

Each function of the learning device 1 and the prediction device 4 is realized by reading predetermined computer software (including a training program and a prediction program) from the main storage device such as the RAM 102 or the auxiliary storage device 107 and executing the computer software by the CPU 101 to read and write in the main storage device such as the RAM 102 or/and the auxiliary storage device 107 and the like and operate the input device 104, the output device 105, and the communication module 106.

Therefore, each part of the learning device 1 and the prediction device 4 illustrated in FIGS. 1 and 11 is realized by cooperation of software and hardware by a processor executing predetermined computer software (including a training program and a prediction program) stored in advance in a computer including the learning device 1 and the prediction device 4.

A computer program implementing at least some of the functions of the parts of the learning device 1 and the prediction device 4 illustrated in FIGS. 1 and 11 may be installed in a storage of one or more computers. The CPU 101 of one or more computers may read a computer program installed in the central processing unit into a main memory and execute the computer program to thereby exert the functions of the respective parts of the prediction device 10 illustrated in FIG. 4.

The learning device 1 and the prediction device 4 illustrated in FIGS. 1 and 11 may be implemented by one or more CPUs 101. Herein, the CPU 101 may refer to one or more electronic circuits disposed on one chip, or may refer to one or more electronic circuits disposed on two or more chips or two or more devices. In the case where multiple electronic circuits are used, each electronic circuit may communicate by wired or wireless means.

The functions of the parts of the learning device 1 and the prediction device 4 illustrated in FIGS. 1 and 11 may be executed by one computer or may be executed by a plurality of computers in a distributed manner. In the case where the functions of the respective parts of the prediction device 4 illustrated in FIG. 4 are executed in a distributed manner by a plurality of computers, the plurality of computers may transmit and receive data via a communication network including a local area network (LAN), a wide area network (WAN), a personal area network (PAN), or the Internet.

The training program and the prediction program can be stored in, for example, the main storage device or the auxiliary storage device 107 included in the computer. The training program and the prediction program may be stored in a computer connected to a communication line such as the Internet, and a part or all of the training program and the prediction program may be downloaded via the communication line. Furthermore, the training program and the prediction program may be configured to be provided or distributed via a communication line.

The training program and the prediction program may be recorded in (or installed on) the computer from a state where a part or all of the prediction program is stored in a portable storage medium such as an optical disk such as a CD-ROM and a DVD-ROM, a semiconductor memory such as a flash memory, or the like.

### <Training Method>

Next, a training method according to the present embodiment will be described. The training method according to the present embodiment is a method for generating a trained model for predicting a feature indicating an intermolecular space between molecules of interest by using a training data set in which molecules of interest (an explanatory variable) and a feature indicating an intermolecular space between molecules of interest in a simulation cell having a freely chosen parallelepiped shape (an objective variable) are associated with each other in a learning device 1 having the configuration illustrated in FIG. 1.

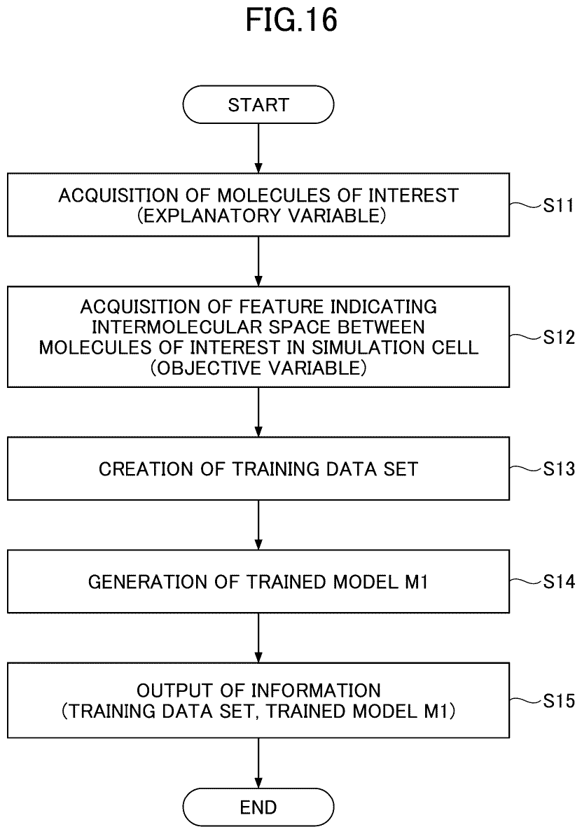

FIG. 16 is a flowchart illustrating the training method according to the present embodiment. As illustrated in FIG. 16, the first acquirer 11 acquires information regarding molecules of interest included in a base oil from the first storage 21 as an explanatory variable (the first acquisition step, step S11).

Next, the second acquirer 12 acquires, as an objective variable, a feature indicating an intermolecular space between molecules of interest in a simulation cell having a freely chosen parallelepiped shape from the second storage 22 (the second acquisition step, step S12).

Next, the training data set creator 13 extracts molecules of interest as an explanatory variable and a feature indicating an intermolecular space between the molecules of interest in a simulation cell having a freely chosen parallelepiped shape as an objective variable, and adds these variables to the training data set (the training data set creation step, S13).

The training data set creator 13 creates a training data set by linking the input molecules of interest and the input feature indicating an intermolecular space between the molecules of interest to each other.

Next, the training part 14 generates a trained model M1 through training using a training data set in which molecules of interest (an explanatory variable) and a feature indicating an intermolecular space between the molecules of interest in a simulation cell having a freely chosen parallelepiped shape (an objective variable) are associated with each other (the training step, step S14).

The training part 14 generates a trained model M1 in accordance with an input of information regarding molecules of interest included in the training data set so that a feature indicating an intermolecular space between molecules of interest in a simulation cell having a freely chosen parallelepiped shape becomes an output.

Next, the outputter 15 outputs information on the training data set used in learning of the trained model M1 and information on the trained model M1 through a form of display or the like (the output step, step S15).

The training method according to the present embodiment includes a training step (step S14), and in the training step (step S14), a trained model M1 that predicts a feature indicating an intermolecular space between molecules of interest in a simulation cell having a freely chosen parallelepiped shape can be generated. In the training method according to this embodiment, a feature indicating an intermolecular space between molecules of interest is predicted from information regarding molecules of interest that are input can be predicted through a use of the trained model M1 generated in the training step (step S14). The feature indicating an intermolecular space between molecules of interest can be used as an index indicating a degree of density of an oil film formed of a base oil composed of the molecules of interest. Therefore, the training method according to the present embodiment is used for predicting a degree of density of an oil film formed of a base oil composed of the molecules of interest that are input, and therefore, the time required for selecting a base oil that brings out an effect of an additive can be shortened.

In the training method according to this embodiment, a feature indicating an intermolecular space between molecules of interest can be predicted from information regarding molecules of interest through use of the trained model M1 generated in the training step (step S14). By using this feature for predicting a degree of density of an oil film formed of a base oil composed of molecules of interest, the burden and time required for predicting a degree of density of an oil film can be reduced. Therefore, the training method according to the present embodiment can reduce the burden in predicting a degree of density of an oil film composed of molecules of interest.

The training method according to the present embodiment can use, as a feature, a value calculated based on a radial distribution function representing the relationship between an atomic distance between atoms of different molecules and an abundance ratio of one molecule to another molecule, of a liquid optimized structure obtained by stabilizing a liquid structure created from a freely chosen number of three-dimensional structures of molecules of interest within a simulation cell. Thus, as the training method according to the present embodiment can use a feature indicating an intermolecular space between molecules of interest for performing prediction with higher accuracy, it is therefore possible to improve the accuracy in selecting the base oil that brings out the effect of the additive.

### <Prediction Method>

Next, a prediction method according to the present embodiment will be described. The prediction method according to the present embodiment is a method in which the prediction device 4 having the configuration illustrated in FIG. 11 predicts, from molecules of interest, the feature indicating an intermolecular space between molecules of interest as an index indicating a degree of density of an oil film including the molecules of interest by the trained model M2.

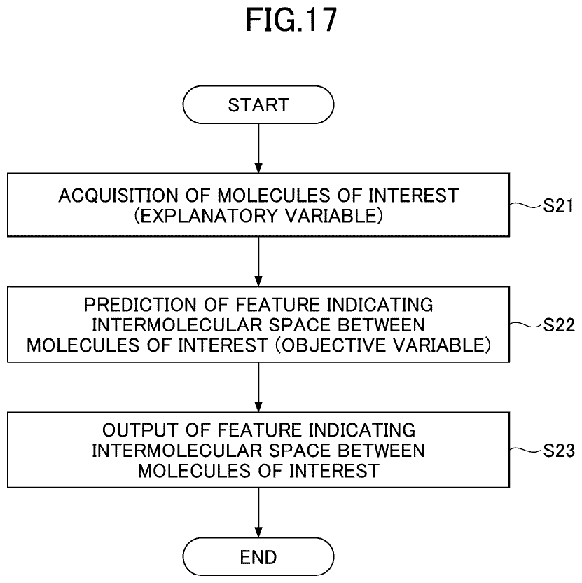

FIG. 17 is a flowchart illustrating the prediction method according to the present embodiment. As illustrated in FIG. 17, molecules of interest included in a base oil, which is a prediction target, are acquired as an explanatory variable by the acquirer 41 (the acquisition step, step S21).

Next, the predictor 42 inputs the molecules of interest as a prediction target acquired by the acquirer 41 to the trained model M2, thereby predicting a degree of density of an oil film formed of a base oil composed of the molecules of interest as a prediction target predicted by the trained model M2 (the prediction step, step S22).

In other words, a feature indicating an intermolecular space between the molecules of interest as a prediction target, which is predicted by the trained model M2, is output as an objective variable by the predictor 42 inputting the molecules of interest as a prediction target, which is acquired by the acquirer 41, to the trained model M2. The objective variable is output by the predictor 42 as an index indicating a degree of density of an oil film formed of a base oil composed of the molecules of interest as a prediction target.

Next, the outputter 43 outputs a feature indicating an intermolecular space between molecules of interest predicted by the trained model M2 as an objective variable in the prediction step S22, and outputs, in a form of display or the like, the feature as a value indicating a degree of density of an oil film formed of a base oil composed of molecules of interest (the output step, step S23).

The prediction method according to this embodiment includes a prediction step (step S22), and in the prediction step (step S22), a feature indicating an intermolecular space between molecules of interest can be predicted from molecules of interest by the trained model M2. In the prediction method according to the present embodiment, a degree of density of an oil film can be predicted by predicting a feature indicating an intermolecular space between molecules of interest in the prediction step (step S22), and therefore, how easily an additive can pass through the oil film can be predicted. In the prediction method according to the present embodiment, the use of the trained model M2 in the prediction step (step S22) makes it possible to predict a degree of density of an oil film in a shorter time and in a simpler manner. Therefore, the prediction method according to the present embodiment can predict, in a short time, a feature indicating an intermolecular space between molecules of interest that is obtained from molecules of interest that are input as an index indicating a degree of density of an oil film, and therefore, time required for selecting a base oil which brings out an effect of an additive can be shortened.

In the prediction method according to the present embodiment, the feature indicating an intermolecular space between molecules of interest is predicted in the prediction step (step S22), thereby reducing the burden and time required for predicting the feature indicating an intermolecular space between molecules of interest. Therefore, the prediction method according to the present embodiment can efficiently and simply predict a degree of density of an oil film formed of a base oil composed of molecules of interest, and therefore, the burden in predicting a degree of density of an oil film formed of a base oil composed of molecules of interest can be reduced.

Although the embodiments have been described above, the embodiments are presented as examples, and the present invention is not limited by the embodiments. The above embodiments can be implemented in various other forms, and various combinations, omissions, substitutions, changes, and the like can be made without departing from the gist of the invention. These embodiments and modifications thereof are included in the scope and gist of the invention, and are included in the invention described in the claims and the scope of equivalents thereof.

The embodiments of the present invention are as follows, for example.
<1> A learning device comprising:
   a training part configured to generate a trained model using a training data set in which molecules of interest, which are molecules included in a base oil that is liquid at normal temperature of 20° C. and normal pressure of 1.013×10⁵ Pa, and a feature indicating an intermolecular space between the molecules of interest in a simulation cell having a freely chosen parallelepiped shape are associated with each other.
<2> The learning device described in <1>, wherein
   when a liquid optimized structure is acquired, the liquid optimized structure being obtained by relaxing a liquid structure created from a freely chosen number of three-dimensional structures of the molecules of interest arranged in the simulation cell, the feature is calculated by calculating an atomic distance between atoms of different molecules from the liquid optimized structure and calculating a radial distribution function representing an abundance ratio of one molecule to another molecule at each atomic distance.
<3> A trained model generated using a training data set in which molecules of interest, which are molecules included in a base oil that is liquid at normal temperature of 20° C. and normal pressure of 1.013×10⁵ Pa, and a feature indicating an intermolecular space between the molecules of interest in a simulation cell having a freely chosen parallelepiped shape are associated with each other.
<4> A prediction device comprising: <4> an acquirer configured to acquire molecules included in a base oil that is liquid at normal temperature of 20° C. and normal pressure of 1.013×10⁵ Pa as molecules of interest; and
   a predictor configured to predict a degree of density of an oil film formed of the molecules of interest using a training data set in which a trained model generated using the molecules of interest and a feature indicating an intermolecular space between the molecules of interest in a simulation cell having a freely chosen parallelepiped shape are associated with each other.
<5> A prediction program for causing a computer to execute:
   an acquisition step of acquiring molecules included in a base oil that is liquid at normal temperature of 20° C. and normal pressure of 1.013×10⁵ Pa as molecules of interest; and
   a prediction step of predicting a degree of density of an oil film formed of the molecules of interest using a training data set in which a trained model generated using the molecules of interest and a feature indicating an intermolecular space between the molecules of interest in a simulation cell having a freely chosen parallelepiped shape are associated with each other,
<6> A prediction method in which a computer is configured to execute:
   an acquisition step of acquiring molecules included in a base oil that is liquid at normal temperature of 20° C. and normal pressure of 1.013×10⁵ Pa as molecules of interest; and
   a prediction step of predicting a degree of density of an oil film formed of the molecules of interest using a training data set in which a trained model generated using the molecules of interest and a feature indicating an intermolecular space between the molecules of interest in a simulation cell having a freely chosen parallelepiped shape are associated with each other.

This application claims priority based on Japanese Patent Application No. 2022-199613 filed with the Japan Patent Office on Dec. 14, 2022, the entire contents of which are incorporated herein by reference.

### REFERENCE SIGNS LIST

1 Learning device
4 Prediction device
11 First storage
12 Second storage
13 Training data set creator
14 Training part
15, 43 Outputter
41 Acquirer
42 Predictor
M1, M2 Trained model

1. A learning device comprising:
a training part configured to generate a trained model through training using a training data set in which molecules of interest, which are molecules included in a base oil that is liquid at normal temperature of 20° C. and normal pressure of 1.013× 10⁵ Pa, and a feature indicating an intermolecular space between the molecules of interest in a simulation cell having a freely chosen parallelepiped shape are associated with each other.

2. The learning device according to claim 1, wherein
when a liquid optimized structure is acquired, the liquid optimized structure being obtained by relaxing a liquid structure created from a freely chosen number of three-dimensional structures of the molecules of interest arranged in the simulation cell, the feature is calculated by calculating an atomic distance between atoms of different molecules from the liquid optimized structure and calculating a radial distribution function representing an abundance ratio of one molecule to another molecule at each atomic distance.

3. A trained model generated using a training data set in which molecules of interest, which are molecules included in a base oil that is liquid at normal temperature of 20° C. and normal pressure of 1.013× 10⁵ Pa, and a feature indicating an intermolecular space between the molecules of interest in a simulation cell having a freely chosen parallelepiped shape are associated with each other.

4. A prediction device comprising:
an acquirer configured to acquire molecules included in a base oil that is liquid at normal temperature of 20° C. and normal pressure of 1.013× 10⁵ Pa as molecules of interest; and
a predictor configured to predict a degree of density of an oil film formed of the molecules of interest using a trained model generated using a training data set in which the molecules of interest and a feature indicating an intermolecular space between the molecules of interest in a simulation cell having a freely chosen parallelepiped shape are associated with each other.

5. A non-transitory storage medium storing a prediction program for causing a computer to execute:
acquiring molecules included in a base oil that is liquid at normal temperature of 20° C. and normal pressure of 1.013× 10⁵ Pa as molecules of interest; and
predicting a degree of density of an oil film formed of the molecules of interest using a trained model generated using a training data set in which the molecules of interest and a feature indicating an intermolecular space between the molecules of interest in a simulation cell having a freely chosen parallelepiped shape are associated with each other.

6. A prediction method in which a computer is configured to execute:
acquiring molecules included in a base oil that is liquid at normal temperature of 20° C. and normal pressure of 1.013× 10⁵ Pa as molecules of interest; and
predicting a degree of density of an oil film formed of the molecules of interest using a trained model generated using a training data set in which the molecules of interest and a feature indicating an intermolecular space between the molecules of interest in a simulation cell having a freely chosen parallelepiped shape are associated with each other.
